(19) United States
(12) Patent Application Publication
(10) Pub. No.: US 2023/0129746 A1
(43) Pub. Date: Apr. 27, 2023

(54) COGNITIVE LOAD PREDICTOR AND DECISION AID

(71) Applicant: TOYOTA RESEARCH INSTITUTE, INC., LOS ALTOS, CA (US)

(72) Inventors: SHABNAM HAKIMI, Chapel Hill, NC (US); YUE WENG, San Mateo, CA (US)

(21) Appl. No.: 17/507,505

(22) Filed: Oct. 21, 2021

(51) Int. Cl. G16H 20/70; G16H 10/20; A61B 5/16; G06F 3/16; G06N 20/00

(52) U.S. Cl. CPC G16H 20/70; G16H 10/20; A61B 5/165; G06F 3/167; G06N 20/00

(57) ABSTRACT

Systems and methods are disclosed for receiving biometric sensor data and generating a first a signal based on one or more conversational prompts. Systems and methods disclosed herein generate a prediction of a cognitive state for a user based on the biometric sensor data, and generate a recommendation based on the predicted cognitive state for the user. Systems and methods disclosed herein can generate, for a user interface, an indication of the generated recommendation.

### TECHNICAL FIELD

The present disclosure relates generally to advanced decision making aids, and smart devices, and more particularly to methods and systems for providing recommendations based on cognitive load or cognitive stress.

### BACKGROUND OF THE INVENTION

Decision making is affected by cognitive or physical stress. Biometric sensors can be used to monitor various cognitive and/or physical conditions of users. Recommendation systems, by providing various suggestions to users, provide recommendations for what user may be interested in.

### BRIEF SUMMARY OF THE DISCLOSURE

As previously alluded to, a person's cognitive state can be detected and is particularly relevant to decisions people make. A cognitive state can include an individual's beliefs, desires, intentions, knowledge, state of being (e.g. whether distracted, uncertain, happy, confused, frustrated, agitated, confident, reclusive, confident, engaged, encouraged, willing to please, interested, bored, tired). Aspects of the present disclosure allow users to lessen the cognitive load of decision making, by providing one or more recommendations to user(s) based on a predicted cognitive state of the user(s).

Methods are described herein for detecting a cognitive state of a user. Methods disclosed herein can be executed at least at a connected device. Various methods can include receiving, by a perception circuit comprising at least one sensor, at least one biometric sensor data. Various methods can include generating a first signal for a user interface. The signal can be based on one or more conversational prompts. Some methods can include generating, by a processing component, a prediction of a cognitive state for a user based on the at least one biometric sensor data. Some methods can include generating a recommendation based on the predicted cognitive state for the user. Example methods can include providing, a second signal for the user interface, the second signal comprising an indication of the generated recommendation. The recommendation can be a common recommendation for multiple users, based on predictions of respective cognitive states for multiple users. The recommendation can include a subset of a set of options. The set of options can include possible operational configurations of a device.

Methods disclosed herein can further include generating a control signal. The control signal configured to control an operation of a device based on the generated recommendation. The control signal can be based on a type of device.

The user interface can include a vocalization circuit. The method can include receiving, by the user interface, a response to the one or more conversational prompts. In some embodiments, the cognitive state for the user is further generated based on the content of the received response.

Methods disclosed herein can include receiving, by the perception circuit, which can include at least one sensor, a second biometric sensor data. Methods disclosed herein can include updating a cognitive state machine learning model based on the second biometric sensor data and the generated recommendation.

Methods disclosed herein can further include receiving a user identifier. In some examples, the prediction is generated, at the generating step of various methods, if the user identifier matches the biometric sensor data.

Various systems are described herein for detecting a cognitive state of a user. Systems disclosed herein can include at least one memory, the at least one memory storing machine-executable instructions. Systems disclosed herein can include at least one processor. Systems disclosed herein can include at least one connected device.

The at least one processor can be configured to access the at least one memory and execute the machine-executable instructions to perform a set of operations.

The set of operations can include operations to detect an availability of a camera based sensor. The set of operations can include generating a first a signal for a user interface, the signal based on one or more conversational prompts. In some example systems, if the availability indicates a camera based sensor is available, the set of operations can include determining the cognitive state of a user based on features extracted from signals from the camera based sensor. In some example systems, if the availability indicates a camera based sensor is not available, the set of operations can include receiving, by at least one other sensor, at least one biometric sensor data.

In various systems, the set of operations can include generating a prediction of a cognitive state for a user based on the received at least one biometric sensor data. The prediction can be generated by a processing component. The set of operations can include generating a recommendation based on the predicted cognitive state for the user.

The set of operations, can include providing a second signal for the user interface. The second signal can include an indication of the generated recommendation.

In some systems, if the availability indicates the camera based sensor is available, the first signal for the user interface include a video conversational prompt. In example systems, if the availability indicates the camera based sensor is not available, the first signal for the user interface can include an audio based vocalized question.

In some embodiments of systems, the recommendation can include a subset of a set of options. The set of options can include possible operational configurations of an operational component of the system.

The operational component of the system can include at least one of, a) a scheduling component, b) a home appliance operational controller, or c) a navigation system.

In some embodiments, systems can include an operational component. The operational component can be configured with two or more operational configurations. The at least one processor can access the at least one memory and execute the machine-executable instructions to generate a control signal, the control signal configured to control an operation of the operational component according to a subset of the two or more operational configurations based on the generated recommendation.

In some systems, the recommendation can be a common recommendation for multiple users. In some embodiments, the recommendation can be based on predictions of respective cognitive states for multiple users.

The set of operations can include receiving a response input signal based on a user response to the one or more conversational prompts. In some embodiments, the cognitive state for the user is further generated based on the response input signal.

In some embodiments, the predicted cognitive state for the user was generated based on a cognitive state machine learning model. The set of operations can include receiving subsequent biometric sensor data from the at least one other sensor. The set of operations can include updating a cognitive state machine learning model based on the subsequent biometric sensor data and the generated recommendation.

The set of operations can include receiving a user identifier. In embodiments of systems, the predicted cognitive state is only generated if the user identifier matches the biometric sensor data.

In some example systems, a set of operations can be performed by at least one processor, accessing at least one memory storing machine-executable instructions, to receive, by a perception circuit comprising at least one sensor, at least one biometric sensor data. The set of operations can include generating a first a signal for a user interface, the signal based on one or more conversational prompts. The set of operations can include generating, by a processing component, a prediction of a cognitive state for a user based on the received at least one biometric sensor data.

In some example systems, the set of operations can include generating a recommendation based on the predicted cognitive state for the user. The set of operations can include providing, a second signal for the user interface, the second signal including an indication of the generated recommendation. The recommendation can be a subset of a set of options. The set of options can be possible operational configurations of a device.

In example systems, the set of operations can include generating a control signal, the control signal configured to control an operation of a device based on the generated recommendation. In some example systems, the recommendation is based on predictions of respective cognitive states for multiple users. The recommendation can be a common recommendation for, or different for respective users of the multiple users.

### BRIEF DESCRIPTION OF THE DRAWINGS

The present disclosure, in accordance with one or more various embodiments, is described in detail with reference to the following figures. The figures are provided for purposes of illustration only and merely depict typical or example embodiments.

FIG. 1 shows an example system having a cognitive load predictor and decision aid according to aspects of the present disclosure.

FIG. 2 illustrates an example architecture for detecting the cognitive state of a user and providing one or more recommendations as described herein.

FIG. 3A illustrates example cloud or networked computing environment and various connected devices for detecting the cognitive state of a user, in accordance with one or more embodiments herein.

FIG. 3B illustrates example connected devices that aspects of the present disclosure can be executed, in accordance with one or more embodiments herein.

FIG. 4A illustrates contributions from sources for creation of a cognitive state, including via biometric sensors, and/or via a conversation agent according to various embodiments described herein.

FIG. 4B illustrates example cognitive states according to various embodiments described herein.

FIG. 5 illustrates example graphical user interface for a user device, in accordance with one or more implementations.

FIG. 6A shows an example method for cognitive load detection and decision aiding according to various embodiments described herein.

FIG. 6B shows yet another example method for cognitive load detection and decision aiding according to various embodiments described herein.

The figures are not exhaustive and do not limit the present disclosure to the precise form disclosed.

### DETAILED DESCRIPTION

As alluded to above, decision making is made more difficult when an individual is cognitively or physically stressed. This difficulty can be exacerbated when an individual is presented with a large number of options, some of which may not be appropriate for their state (e.g. cognitive or emotional). Individuals are faced with multiple decisions throughout the day and can face decision fatigue which is compounded by multiple other cognitive and/or emotional stressors. The invention is designed to augment smart devices that present users with one or more options for the user, and tailor choice options and recommendations to the user's current cognitive state (e.g. cognitive load or stress). Aspects of the present disclosure can be executed at one or more smart (i.e. network-connected) devices, such as household devices (such as cleaning appliances, smart closets, kitchen appliances such as coffee machines, ovens, or refrigerators, lighting systems, doorbells, TVs, media devices, massage equipment, chairs or couches), industrial devices (e.g. robotic equipment, additive and/or subtractive manufacturing systems), design equipment, personal grooming or hygiene devices (e.g. pools, spas, toothbrush, styling devices, automatic nail painting, hair-cut, make-up devices), mobility devices (e.g. vehicles, scooters, bicycles), networking systems (e.g. transportation systems for selecting transportation, personal/friendship/workplace networking systems), commercial devices (such as vending machines, robotic kitchens, cake decorating machines), workplace devices (such as scheduling systems, collaborative work systems, space planning and/or design systems), and/or educational devices (such as lesson or training planning systems).

A cognitive state detection circuit as described herein can predict a cognitive state of the user. A recommendation circuit can be configured to provide one or more personalized recommendations for the user based on the predicted cognitive state.

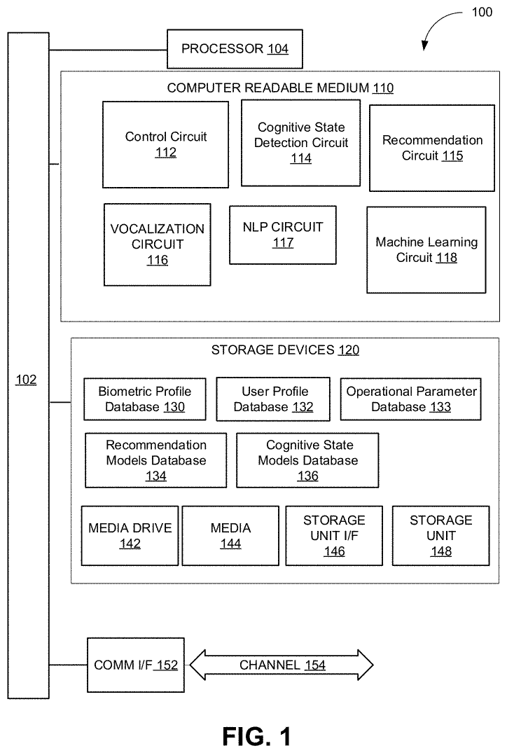

FIG. 1 shows an example system 100 having a cognitive load predictor and decision aid according to aspects of the present disclosure. System 100 can be implemented as or as part of a smart device. System 100 can include a bus 102 or other communication mechanism for communicating information. However, any communication medium can be used to facilitate interaction with other components of system 100.

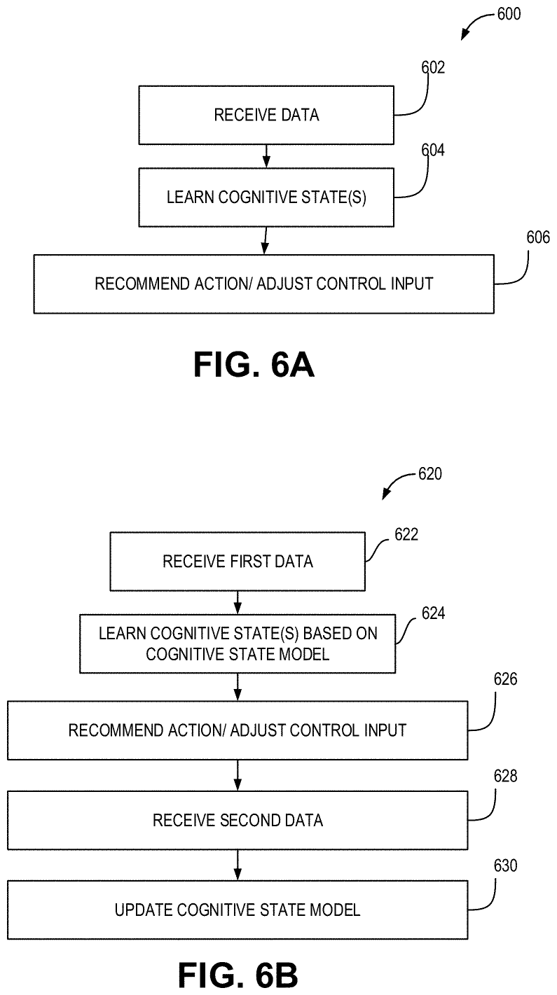

System 100 can include one or more processor 104 coupled with bus 102 for processing information. As such, system 100 can include a computing component. Processor 804 might be implemented using a general-purpose or special-purpose processing engine such as, for example, a microprocessor, controller, or other control logic. The processor might be specifically configured to execute one or more instructions for execution of logic of one or more circuits described herein. In embodiments, processor 104 may fetch, decode, and/or execute one or more instructions to control processes and/or operations for enabling aspects of the present disclosure. For example, instructions can correspond to steps for performing one or more steps of method 700 shown in FIG. 7. Processor 104 can be a hardware processor. Processor 104 can include one or more GPUs, CPUs, microprocessors or any other suitable processing system. Processor 104 may include one or more single core or multicore processors. Processor 104 can execute one or more instructions stored in a non-transitory computer readable medium 110. Computer readable medium 110 can be a main memory, and/or other auxiliary memory, such as a random access memory (RAM), cache and/or other dynamic storage device(s). For example, random access memory (RAM) or other dynamic memory, might be used for storing information and instructions to be fetched, decoded, and/or executed by processor 104. Such instructions may include one or more instructions for execution of methods 600, 620 described below with reference to FIG. 6A and FIG. 6B and/or for execution of one or more logical circuits described herein. Memory also be used for storing temporary variables or other intermediate information during execution of instructions to be fetched, decoded, and/or executed by processor 104. might likewise include a read only memory (“ROM”) or other static storage device coupled to bus 802 for storing static information and instructions for processor 804.

Computer readable medium 110 can contain one or more logical circuits. Logical circuits can include one or more machine-readable instructions which can be executable by processor 104 and/or another processor. Logical circuits can include one or more instruction components. Instruction components can include one or more computer program components. For example, control circuit 112, cognitive state detection circuit 114, recommendation circuit 115, vocalization circuit 116, natural language processing circuit 117, and/or machine learning circuit 118. At least one of these logical circuits (and/or other logical circuits which are not shown) can allow for predicting the current the cognitive state of one or more users and contextualizing the current state in the one or more users' past behavior and preferences. At least one of these logical circuits (and/or other logical circuits) can recommend one or more of a selection for the user(s) based on the detected cognitive state.

As previously alluded to, aspects of the present disclosure can be executed at one or more devices. Control circuit 112 can be configured to perform one or more primary controls for the system 100. Aspects of control circuit 112 may depend on the type of device(s) the system 100 is integrated into. For example, with reference to a smart device, the control circuit 112 can be configured to control one or more aspects of the smart device. For example, if the smart device is a kitchen appliance (e.g. toaster, coffee machine, refrigerator), the control circuit 112 can be configured to control elements of the kitchen appliance for performing one or more functions. With reference to a coffee machine, control circuit 112 may be able to control the style of brewing, the size of coffee grind, the type of coffee roast, the selection of beans, the temperature of the coffee brew, etc. For example, control circuit 112 may be able to generate one or more actuation signals, for example for actuation of one or more flow valves, and/or trigger one or more heating elements of the coffee machine. With reference to smart closets, control circuit 112 may be able to generate one or more actuation signals for moving one or more garments to a location within the smart closet based on a selection of an outfit or garment.

With reference to scheduling systems, the control circuit 112 can be configured to generate one or more meeting invitations or otherwise schedule events, book rooms, etc. With reference to navigation systems, one or more waypoints, directions, or navigations can be provided, and various options thereof can be controlled by control circuit 112. With reference to a vehicle, control circuit 112 can be configured to operate of one or more components of vehicle, such as sensors, computing system, autonomous vehicle control systems, and/or other vehicle systems. Control circuit 112 may be able to operate one or more controls for the system based on one or more recommendations from the recommendation circuit 115, and/or one or more user inputs. It can be understood that recommendation circuit 115 can output one or more recommendations, suggestions, messages, and/or prompts, and/or the control circuit 112 can control one or more aspects of the system based on recommendation. It can be understood that the control circuit 112 can generate one or more control inputs for the system based on the recommendation. It can also be understood that the recommendation from recommendation circuit 115 can include one or more options for selection by a user, and the control circuit 112 can generate a control signal for the system based on the user selection.

Control circuit 112 can contain and/or or operate one or more control algorithms and/or models. Control algorithms can allow for automating one or more sensors(s) (e.g. biometric sensors or selection confirmations sensor), and or aspects of the control system (e.g. actuators), so that the system can perform one or more designated operations.

Cognitive state detection circuit 114 can detect past or current cognitive state of one or more users, and predict the current or future cognitive state of the one or more users. Cognitive state detection circuit 114 can include sensors or receive information from other elements of the system 100, such as storage device 120, and/or from other systems 100 or infrastructure. The cognitive state detection circuit 114 can utilize information about the current context of the user (e.g., time of day) as well as user's past behavior and preferences while using the system 100 (e.g. aspects of the connected smart device) to detect the users current level of cognitive load. For example, contextual information relevant for recommending a type of coffee (i.e. by recommendation circuit 115), can include information about the time of morning at which a pot of coffee is brewed, or the volume of coffee brewed, as applied to the cognitive state of the user. In other words, the cognitive state can be based on one or more detected cognitive states (e.g. by biometric sensors) and/or predicted cognitive states of the user. Cognitive states can be detected, for example, by camera based sensors (e.g. by computer vision) based on face, eye, and body features extracted from the image and/or video. The predicted cognitive state can be based on at least one detective cognitive state, and/or contextual information. In some embodiments, the cognitive state can be detected and/or predicted based on a conversational agent implemented at least at vocalization circuit 116 and/or NLP circuit 117. Cognitive state detection circuit 113 can include a sensor fusion circuit and/or machine perception circuit, computer vision circuit, for determining the cognitive state of the user from values of parameters from one or more sensors (or other devices/elements of the system). Sensor fusion can allow for evaluating data from the plurality of sensors. Sensor fusion circuit (not shown) can execute algorithms to assess inputs from the various sensors.

Vocalization circuit 116 be coupled to one or more user interfaces, such as a graphical user interface (e.g. for text/image/video based user interaction) or speaker. The conversational agent can be configured to engage in a dialogue with the user. In some embodiments, the dialogue can include one or more, two or more, three or more, five or more, ten or more back and forth questions between the user and the system. The questions can be direct (i.e. directly asking for the cognitive state of the user), and/or passive/circumstantial. For example, the user's cognitive state can be established from the user's response to one or more questions aimed at understanding aspects of the user's cognitive state.

The system will leverage natural language processing techniques to analyze a user's cognitive state based on their speech, extracting voice and semantic features. from the conversation. The data from the camera and the built-in conversational agent may also be combined to enhance cognitive state detection and subsequent recommendations.

Cognitive state detection circuit 114 can contextualizing the current state in the one or more users' past behavior and preferences and generate one or more contextual information related to (and/or mapped to) aspects of the cognitive state(s). The contextual information may include information regarding the surrounding contextual environment of the system 100 or the user, including other devices (such as in the case of vehicles and/or obstacles). The contextual information can include one or more objects and/or features of a surrounding environment to the system 100. Contextual information can include one or more aspects of the surrounding environment that can affect the cognitive state of the user. Contextual information can include one or more proximal (spatially and/or temporally) aspects of the user's life. For example, contextual information can be gathered from a user's agenda, work system, mail systems, social networks, etc. Contextual information can be gathered from one or more other systems 100. Contextual information can include who or what the user is or was interacting with. Contextual information can be determined from sensors of the device.

With respect to systems as part of vehicles, determination of the contextual information may include identifying obstacles, identifying motion of obstacles, estimating distances between the vehicle and other vehicles, identifying lane markings, identifying traffic lane markings, identifying traffic signs and signals, identifying crosswalk indicators, identifying upcoming curvature of the roadway, and/or other determinations. Determination of the contextual information may include identification of ambient conditions such as other individuals proximate to the system, traffic, temperature, rain, snow, hail, fog, and/or other ambient conditions that may affect the cognitive state of the user.

Recommendation circuit 115 can be implemented as a cognitive state dependent recommendation engine. Based on the detected cognitive load (e.g. by cognitive state detection circuit 114) the system can recommend one or more of a plurality of options or actions accordingly. The recommendation can depend on the device and/or type of device. For example, a coffee machine could recommend a stronger or milder coffee, a specific temperature of coffee (hot/cold), a style of preparation (e.g. with milk/sugar/foam), a specific roast of coffee, and/or a specific volume of coffee, based on the detected cognitive state of the user. In another example, a selection from a vending machine can be recommended based on the cognitive state of the user. In another example implemented in a scheduling system, a meeting can be scheduled at a mutually convenient (for the cognitive states) of users, as well as contextually convenient (e.g. based on the complexity of the subject matter for the meeting and/or other availabilities). In some embodiments, a smart closet can select a wardrobe and/or article of clothing for a user. In some embodiments in vehicular contexts, waypoints (e.g. gas station, restaurant) or routes (e.g. low stress, low traffic, high entertainment value) can be recommended. For example, a driver for a vehicle may be suggested specific routes (and/or clients for pick-up/drop-off in a taxi or delivery context) based on the driver's cognitive state. In some examples, lesson plans can be arranged, or test question can be administered, based on the cognitive state of the learners.

As previously alluded to, individuals are faced with multiple decisions throughout the day. Individuals may face decision fatigue, and decision fatigue is compounded by multiple other cognitive and/or emotional stressors individuals may face throughout the day. In some example systems 100, the recommendations allows for minimizing a cognitive load of the user.

The system 100 can utilize machine learning to determine the cognitive state of the user (such as by cognitive state detection circuit 114), and/or one or more recommendations for the user (e.g. by recommendation circuit 115). Machine learning circuit 118 can be configured to operate one or more machine learning algorithms. Machine learning algorithms can be used to determine and/or learn the cognitive state of one or more users, and/or one or more recommendations as disclosed herein. For example, a model of a user's cognitive state or cognitive state can be learned by reinforcement learning. Similarly, the model can consider how a user's cognitive state may change, depending on one or more selections. In some embodiments, it may be useful for recommendations to be made that minimize a cognitive load. In some embodiments, data (i.e. values for parameters measured by sensors) are preprocessed (e.g. by filtering (e.g. median filter) e.g. by adaptive artifact removal), and feature extraction and selection is performed. The selected features can be classified (e.g. by one or more classification algorithms).

Machine learning algorithms can be utilized to control aspects of the system, such as by control circuit 112. A cognitive state can be fixed and/or updated (e.g. updated during operation of the control algorithm) parameters, which allow for the vehicle control algorithm to be executed (e.g. by vehicle control circuit 212 and/or another logical circuit). Machine learning circuit 218 can operate one or more machine learning algorithms, and/or deep learning algorithms. For example, such algorithms can be implemented as at least one of a feedforward neural network, convolutional neural network, long short-memory network, autoencoder network, deconvolutional network, support vector machine, inference and/or trained neural network, recurrent neural network, classification model, regression model, etc. Such algorithms can include supervised (e.g. k-NN, support vector machine, Kernel density estimation) unsupervised, and/or reinforced learning algorithms. For example, machine learning circuit 118 can allow for performing one or more learning, classification, tracking, and/or recognition tasks. For example, one or more facial and/or body expressions can be extracted from images and/or video. Machine learning circuit 118 can be trained. The machine learning circuit 118 can be trained by simulating, by one or more logical circuits, across a range of biometric data, cognitive states, across a range of recommendations. The machine learning circuit can be trained by comparing one or more outcomes for the recommendation (e.g. by comparing a predicted cognitive state, to an actual cognitive state, by a performance outcome for the system (e.g. by control circuit 112), by asking the user, or based on contextual information.

System 100 can include one or more storage devices 120. Although a single storage device 120 is shown, it can be understood that storage devices can be multiple elements, and/or be distributed (i.e. over a network and/or over devices). Storage devices 120 can include one or more databases. For example, there can be a biometric profile database 130 and a user profile database 132. As previously alluded to, a user's cognitive state can be multifaceted. The user's biometric profile can include values for one or more parameters that can affect the cognitive state of a user, across one or more dimensions of a cognitive state. The biometric profile database can also include one or more weights for the various parameters. The weights may have learned and/or trained by aspects of the present disclosure. The weights may depend on values for one or more contextual parameters. For example, while someone may be heavily negatively influenced by bad weather, another user may be less affected. As such, the user profile database 132 can store one or more user identifiers. The user profile database can store one or more user preferences (which can be learned and/or identified). The user identifier can be alphanumeric identifier that can be linked to the user's biometric data in the biometric profile database 130. The system 100 can recognize the user and the respective user identifier (e.g. in user profile database 132) based on a recognized biometric data (e.g. in biometric database 130 and/or by cognitive state detection circuit 114). In some embodiments, the user can be recognized by the user identifier or other identifier (such as by multi-factor user authentication). When no biometric sensors are available (for example to recognize the user's biometric data), the user can enter their assigned user identifier (or other ID) into the system using an input device (e.g., touchscreen keypad). When biometric sensing is available, the user is recognized and their biometric information can be linked to their identifier to retrieve their information from a database (e.g. storage device 120, cloud database or memory of the system 100 depending on the setup). The user may also choose a custom identifier if desired.

Biometric profile data can be stored in the biometric profile database 130. In some embodiments, it can be stored temporarily (e.g. until a cognitive state is determined by cognitive state detection circuit 114). Biometric profile data generally refers to body dimension and physical and mental behavior measurement values and calculations, including those obtained from remote devices and sensors (such as cameras), as well as mobile, wearable and sensor-based devices used while in physical contact or in proximity to a user. Biometric profile data may be determined from biometric data and can refer to distinctive, measurable characteristics used to label and describe individual activity, cognitive state and behavioral characteristics, such as related to patterns of behavior, including but not limited to typing, rhythm, gait, and sleep and voice qualities and patterns.

Operational parameter database 133 can include operational information for specific one or more devices, and/or possible ranges for such operational information. In some implementations, operational information can include contextual information for the device, including as determined by or for control circuit 112, and contextual information (and contextual parameters) for the user, including those which may be useful in determining the cognitive state of the user. A controls model for the device as implemented by control circuit 112, can include or more for set parameters and models in the operational parameter database. For example, these can be related to vehicle handling models with respect to vehicle devices, which model how the vehicle will react to certain driving conditions, such as how a tire can react to lateral and/or longitudinal tire forces, or human driver models. Other models can include traffic or weather models, or other environment models which can generally include information for simulating the environment or context. For example, mapping data (such as the locations for mapped features such as roads, highways, traffic lanes, buildings, parking spots, etc.), infrastructure data (such as locations, timing, etc. for traffic signals), or terrain (such as the inclinations across one or more axes for features in the mapping data) can be relevant operational parameters in operational parameter database.

In some implementations, the current cognitive state and/or predicted or future cognitive state can be determined by cognitive state detection circuit 114 may be stored electronic storage 120 and considered a prior cognitive state. As another example of storage device(s) 120, there can be recommendation models database 134 and cognitive state models database 136. These databases 134, 136, can store one or more models, training data, weight, and/or gains for execution of one or more algorithms disclosed herein, and can interface with other elements of storage device 120 and computer-readable medium 110. Recommendation models database 134 can include information necessary for generating a recommendation. For example, the recommendation models database 134 can contain one or more recommendation algorithm (e.g. collaborative filtering), controls algorithms or models, a mapping between cognitive states and one or more possible selections, options, and/or device operations, and associated controls parameters such as weights, gains, and/or biases.

Cognitive state models database 136 can include one or more cognition models, which can model how an individual can react in certain situations, and how one or more cognitive states can adjust. Cognitive state models database can include a mapping of one or more values for aspects of a user's biometric profile to one or more values for dimensions of a cognitive state. Cognitive state models database can include mapping for one or more NLP based indications extracted from conversations with conversational agents described herein, to one or more values for dimensions or contributing factors of a cognitive state. It can be understood that the recommendation can be based on the cognitive state, as such the recommendation model can depend on the cognitive state model. It can also be understood that various cognitive state models can depend on the options available for the recommendation to be selected.). A cognitive state model can include a mapping between values for one or more contributing factors to the cognitive state, the sources for the data, and one or more recommendations. For example, for the same values of contributing factors, the mapping can be different for recommending first type of user action, than recommending second type of user action. In some models, the recommendation can be selected so that a cognitive state is maintained or that a cognitive state is obtained.

It can also be understood that various cognitive states (i.e. current and/or previous in a time-series) can be stored at storage devices 120.

The system 100 may also include one or more various forms of information storage devices 120, which may include, for example, a media drive 142 and a storage unit interface 146. The media drive 142 may include a drive or other mechanism to support fixed or removable storage media 144. For example, a hard disk drive, a floppy disk drive, a magnetic tape drive, an optical disk drive, a CD or DVD drive (R or RW), or other removable or fixed media drive may be provided. Accordingly, storage media 144 may include, for example, a hard disk, a floppy disk, magnetic tape, cartridge, optical disk, a CD or DVD, or other fixed or removable medium that is read by, written to or accessed by media drive 142. As these examples illustrate, the storage media 144 can include a computer usable storage medium having stored therein computer software or data.

In some embodiments, information storage devices 120 may include other similar instrumentalities for allowing computer programs or other instructions or data to be loaded into the system 100. Such instrumentalities may include, for example, a fixed or removable storage unit 148 and an interface 146. Examples of such storage units 148 and interfaces 146 can include a program cartridge and cartridge interface, a removable memory (for example, a flash memory or other removable memory engine) and memory slot, and other fixed or removable storage units 148 and interfaces 146 that allow software and data to be transferred from the storage unit 146 to the system 100.

System 100 may also include a communications interface 152. Communications interface 152 may be used to allow software and data to be transferred between system 100 and another device, and/or an external devices. Examples of communications interface 152 may include a modem or softmodem, a network interface (such as an Ethernet, network interface card, WiMedia, IEEE 102.XX or other interface), a communications port (such as for example, a USB port, IR port, RS232 port Bluetooth® interface, or other port), or other communications interface. Software and data transferred via communications interface 152 may typically be carried on signals, which can be electronic, radio, electromagnetic (which includes optical) or other signals capable of being exchanged by a given communications interface 152. These signals may be provided to communications interface 152 via a channel 154. This channel 154 may carry signals and may be implemented using a wired or wireless communication medium. Some examples of a channel may include a phone line, a cellular link, an RF link, an optical link, a network interface, a local or wide area network, and other wired or wireless communications channels.

In embodiments, communications interface 152 can be used to transfer information to and/or from one or more devices, and/or infrastructure. In some embodiments, some or all information stored in storage devices 120 and computer readable medium 110 can be information retrieved from (or to be provided to) one or more devices.

In some examples, a configuration interface (e.g. via communication interface 152) can be used by administrators to customize the user experience and the type of recommendations provided. For example, the administrator may choose to limit or increase the number of interactions with the conversational agent. In another example, the administrated can update training data for one or more models. Specifically, information in storage devices 120, and/or one the information at one or more logical circuits, by a graphical user interface (GUI) or another interface (e.g. a configuration interface corresponding to a administrative control that can be coupled to communications interface 152).

In some embodiments, one or more data or information or described herein can be displayed on the GUI. In one example implementation, the user selections can be input at the GUI. In some embodiments, user input related to a conversation with conversation agent can be input at the GUI. In some embodiments, the user can interact via microphone coupled to communication interface 152 (e.g. for the conversation with the conversation agent). In some embodiments, a conversation with the conversation agent includes a video based conversation. The text/audio video can be analyzed via speech recognition (e.g. by NLP circuit 117), kinetic and/or biometric parameters of a user (e.g. detection of facial expressions and body movement on video). In some embodiments, the conversation agent and/or biometric sensors can analyze emotions, stress level, and user reactions, expressions, actions, gestures, mental states, physiological data cognitive states, physiological data. Facial expressions can be analyzed, including to identify gestures, smiles, frowns, brow furrows, squints, lowered eyebrows, raised eyebrows, attention, eye movement, blinking, brow lifting, and other facial indicators of expressions. Gestures can also be identified, and can include a head tilt to the side, raised hands, fidgeting, a forward lean, a smile, a frown, as well as many other gestures.

In embodiments, the system 100 can output (e.g. at GUI, over communication interface 152, and/or stored in a storage device 120 e.g. storage media 144) one or more values for a recommendation. In embodiments, the system 100 can receive one or more confirmations of a user selection (e.g. corresponding to if the user followed the recommendation or not). In this and/or in other implementations, the system 100 can output (e.g. at GUI, over communication interface 152, and/or stored in a storage device 120 e.g. storage media 144) one or more training data and/or training data sets. Training data and/or training data sets can include values for one or more cognitive state parameters. Weights, gains, and/or biases for one or more control and/or machine learning algorithms can also be received and/or transmitted.

In some embodiments, one or more results for a recommendation can be displayed on the GUI, so can one or more prompts related to conversation agents described herein. As such, simulation systems 100 as described herein can contain one or more visualization components which allow for the visual (or other) rendering of recommendations, such as a set of options for selection. For example, in the context of a system that allows for selection of a navigation route or waypoint based on a cognitive state, the system 100 may allow for the display of a route to be navigated and/or one or more waypoints. In some embodiments, the control circuit 112 can allow for the system to take control over the device (as compared to control by a user) based on the cognitive state. For example, based on the detected and/or predicted cognitive state (e.g. by cognitive state detection circuit 114), a vehicle can be controlled. In other systems, a shopping list can be created and items from the shopping list can be ordered. In other embodiments, the recommendation circuit 115 can select a mutually convenient based on the cognitive state of user (e.g. between multiple users) menu, and/or meeting time and location. As such, at a GUI, one or more alerts that a selection has been made or control has been taken based on the cognitive state, can be displayed. Further, the system 100 can automatically determine if the recommendation allowed for a specific outcome for the device, however a feedback from a user can be used to confirm (or not) such predicted recommendation.

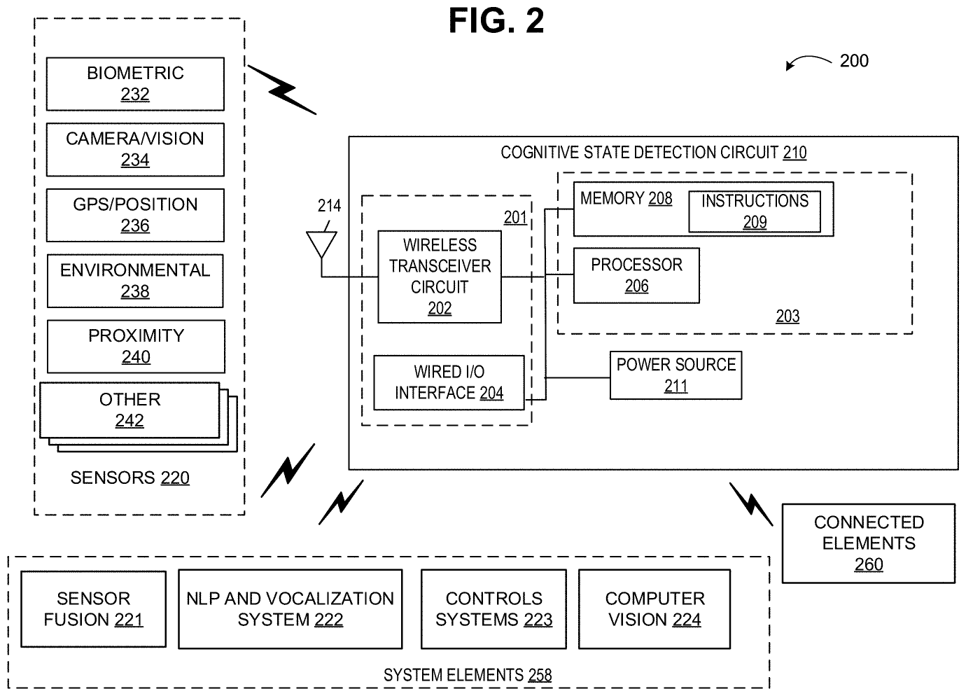

FIG. 2 illustrates an example architecture for detecting the cognitive state of a user and providing one or more recommendations as described herein. Referring now to FIG. 2, in this example, a cognitive state detection and decision aid system 200 includes a cognitive state detection and response circuit 210, a plurality of sensors 220, and a plurality of systems elements 258. Also included are various connected devices and systems 260 with which the cognitive state detection and decision aid system 200 can communicate. System elements 258 can depend on the type of device the cognitive state detection circuit 210 is implemented into. For example, system elements 258 can include one or more previously discussed circuits, e.g. sensor fusion system 221 having a sensor fusion circuit, NLP and vocalization system 222 having NLP 117 and/or vocalization 116 circuits. Systems 258 can include control circuit 112 (with reference to FIG. 1) as part of control system(s) 223. Systems 258 can include computer vision circuit in computer vision system 224. Systems 258 can be configured to detect and/or generate one or more cognitive states for a user, generate a recommendation for a user, and/or control an aspect of the device based on the cognitive state and/or the recommendation. For example, computer vision system 224 can be configured to perform feature extraction based on at least one of a video, image, and/or audio, or other biometric sensor based data. Feature extraction can allow for determination of the cognitive state of a user. For example, face, eye, and/or body features can be extracted from the image and/or video. Features can be learned from embedding models.

As previously alluded to, cognitive state detection and decision aid system 200 can be implemented as and/or include one or more components of one or more devices described herein. With respect to vehicles, circuit 210 can be implemented as an electronic control unit (ECU) or as part of an ECU of a vehicle. In other embodiments, cognitive state detection and response circuit 210 can be implemented independently of the ECU, for example, as another system 258 of the vehicle. Further, with respect to vehicle based devices, sensors 220, system elements 258, and cognitive state detection and response circuit 210 can be part of or include an automated vehicle system/advanced driver assistance system (ADAS). ADAS can provide navigation control signals (e.g. control signals to actuate the vehicle and/or operate one or more systems 258) for the vehicle to navigate a variety of situations. As used herein, ADAS can be an autonomous vehicle control system adapted for any level of vehicle control and/or driving autonomy. For example, the ADAS can be adapted for level 1, level 2, level 3, level 4, and/or level 5 autonomy (according to SAE standard). ADAS can allow for control mode blending (i.e. blending of autonomous and/or assisted control modes with human driver control). ADAS can correspond to a real-time machine perception system for vehicle actuation in a multi-vehicle environment. Continuing the example of a vehicle, controls systems 223 can include controls systems for an ADAS, such as steering controls, throttle/brake controls, transmission control, propulsion control, vehicle hardware interface controls, actuator controls, sensor fusion systems, risk assessment systems, computer vision systems, obstacle avoidance systems, path and planning systems as known in the vehicle arts.

Sensors 220, systems 258, and connected devices and systems 260, can communicate with the cognitive state detection and response circuit 210 via a wired or wireless communication interface. Although sensors 220, system elements 258, and connected devices and systems 260, are depicted as communicating with cognitive state detection and response circuit 210, they can also communicate with each other and/or directly with other devices 260. Data as disclosed herein can be communicated to and from the cognitive state detection and response circuit 210. For example, various infrastructure or devices can include one or more databases, such as of profile data of the user. This data can be communicated to the circuit 210, and can such data can be updated based on the cognitive state of the user. Similarly, the aforementioned contextual information, such as traffic information, vehicle state information (e.g. brake status, steering angle, trajectory, position, velocity), time of day information, demographics, agenda information, or social data for users can be retrieved and updated. Similarly, models, circuits, and predictive analytics can be updated according to various outcomes.

Cognitive state detection and response circuit 210 can generate a cognitive state for a user and generate recommendations for the user based on one or more users cognitive states. As will be described in more detail herein, the cognitive state of a user can be determined based on one or more parameters. Various sensors 220, systems 258, or connected devices or elements 260 may contribute to gathering data for generation of one or more cognitive states of users. For example, the cognitive state and respective recommendation generated by cognitive state detection and response circuit 210 can be generated by one or more circuits (see circuits of FIG. 1).

Cognitive state detection and response circuit 210 in this example includes a communication circuit 201, a decision and control circuit 203 (including a processor 206 and memory 208 in this example), a power source 211 (which can include power supply) and cognitive state detection and response circuit 210. It is understood that the disclosed cognitive state detection and response circuit 210 can be compatible with and support one or more standard or non-standard protocols. Although circuits herein (e.g. circuit 210) are illustrated as a discrete computing system, this is for ease of illustration only, and circuit 210 and other circuits (including respective memory and processor(s)) can be distributed among various systems or components.

Components of cognitive state detection and response circuit 210 are illustrated as communicating with each other via a data bus, although other communication in interfaces can be included. Decision and control circuit 203 can be configured to control one or more aspects of detecting one or more cognitive states of user(s) and recommending or taking an action based on the detected cognitive state(s). Decision and control circuit 203 can be configured to execute one or more steps described with reference to FIGS. 7A-7D.

Processor 206 can include a GPU, CPU, microprocessor, or any other suitable processing system. The memory 208 may include one or more various forms of memory or data storage (e.g., flash, RAM, etc.) that may be used to store the calibration parameters, images (analysis or historic), point parameters, instructions and variables for processor 206 as well as any other suitable information. Memory 208, can be made up of one or more modules of one or more different types of memory, and may be configured to store data and other information as well as operational instructions 209 that may be used by the processor 206 to execute one or more functions of cognitive state detection and response circuit 210. Instructions 209 can include instructions for execution of control circuit 112, cognitive state detection circuit 114, recommendation circuit 115, vocalization circuit 116, NLP circuit 117, and/or machine learning circuit 118. For example, data and other information can include received messages, and/or data related to generating one or more observation based models for the road traffic network and for generating one or more hyper-graphs as disclosed herein. Operational instruction 209 can contain instructions for executing logical circuits, and/or methods as described herein.

Although the examples of FIG. 1 and FIG. 2 are illustrated using processor and memory circuitry, as described below with reference to circuits disclosed herein, decision circuit 203 can be implemented utilizing any form of circuitry including, for example, hardware, software, or a combination thereof. By way of further example, one or more processors, controllers, ASICs, PLAs, PALs, CPLDs, FPGAs, logical components, software routines or other mechanisms might be implemented to make up an cognitive state detection and response circuit 210. Components of decision and control circuit 203 can be distributed among two or more decision and control circuits 203, performed on other circuits described with respect to cognitive state detection and response circuit 210, be performed on devices (such as cell phones) performed on a cloud-based platform (e.g. part of infrastructure), performed on distributed mart elements devices, such as at multiple vehicles, smart phones, smart watches, home appliances, user device, central servers, performed on an edge-based platform, and/or performed on a combination of the foregoing.

System 100 (with reference to FIG. 1) and circuit 200 (with reference to FIG. 2) greater or fewer quantity of systems and subsystems and each could include multiple elements. Accordingly, one or more of the functions of the technology disclosed herein may be divided into additional functional or physical components, or combined into fewer functional or physical components. Additionally, although the systems and subsystems illustrated in FIG. 1 and FIG. 2 are shown as being partitioned in a particular way, the functions of system 100 and 200 can be partitioned in other ways. For example, various systems and subsystems (including on separate devices) can be combined in different ways to share functionality.

Communication circuit 201 either or both a wireless transceiver circuit 202 with an associated antenna 214 and a wired I/O interface 204 with an associated hardwired data port (not illustrated). As this example illustrates, communications with cognitive state detection and response circuit 210 can include either or both wired and wireless communications circuits 201. Wireless transceiver circuit 202 can include a transmitter and a receiver (not shown), e.g. a broadcast mechanism, to allow wireless communications via any of a number of communication protocols such as, for example, WiFi (e.g. IEEE 802.11 standard), Bluetooth, near field communications (NFC), Zigbee, and any of a number of other wireless communication protocols whether standardized, proprietary, open, point-to-point, networked or otherwise. Antenna 214 is coupled to wireless transceiver circuit 202 and is used by wireless transceiver circuit 202 to transmit radio signals wirelessly to wireless equipment with which it is connected and to receive radio signals as well. These RF signals can include information of almost any sort that is sent or received by cognitive state detection and response circuit 210 to/from other components of the system, such as sensors 220, systems elements 258, cloud components, infrastructure (e.g. servers cloud based systems), and/or other devices 260. These RF signals can include information of almost any sort that is sent or received by devices. Transmitted data may include or relate to data in storage device 120. Wireless communications circuit 201 may allow the system to receive updates to data that can be used to execute one or more control algorithms (see control circuit 112) to detect the cognitive state of the user(s) (e.g. by cognitive state detection circuit 114), and make one or more recommendations (e.g. by recommendation circuit 115).

Wireless communications circuit 201 may receive data and other information from sensors 220 or other connected devices 260 or infrastructure, that is used in determining the cognitive state of one or more users. Additionally, communication circuit 201 can be used to send an activation signals, control signals or other activation information to various systems 258, for example, based on a recommendation. For example, in the case of a smart coffee machine device, communication circuit 201 can be used to send signals to one or more system elements 258 for brewing of coffee based on a recommendation. In the case of a vehicle, communication circuit 201 can be used to send one or more control signals for actuators of the vehicle based on the recommendation, e.g. with respect to vehicle speed, maximum steering angle, throttle response, vehicle braking, torque vectoring, and so on.

Wired I/O interface 204 can include a transmitter and a receiver (not shown) for hardwired communications with other devices. For example, wired I/O interface 204 can provide a hardwired interface to other components, including sensors 220, and systems 258. Wired I/O interface 204 can communicate with other devices using Ethernet or any of a number of other wired communication protocols whether standardized, proprietary, open, point-to-point, networked or otherwise.

Power source 211 such as one or more of a battery or batteries (such as, e.g., Li-ion, Li-Polymer, NiMH, NiCd, NiZn, and NiH2, to name a few, whether rechargeable or primary batteries), a power connector (e.g., to connect to vehicle supplied power, another vehicle battery, alternator, etc.), an energy harvester (e.g., solar cells, piezoelectric system, etc.), or it can include any other suitable power supply. It is understood power source 211 can be coupled to a power source of the vehicle, such as a battery and/or alternator. Power source 211 can be used to power the cognitive state detection and response circuit 210.

Sensors 220 can include one or more sensors that may or not otherwise be included on standard devices (e.g. vehicles, home appliances, etc.) with which the cognitive state detection and response circuit 210 is implemented. In the illustrated example, sensors 220 include various biometric sensors 232, camera vision based sensors 234, GPS or other position based sensors 236, environmental sensor 238 (e.g. wind, humidity, pressure, weight, vibration), proximity sensors 240, and other sensors 242 (e.g. accelerometers, etc.). Additional other sensors 242 can also be included as may be appropriate for a given implementation of cognitive state detection and decision aid system 200. Example biometric sensors 323 include sensors within smart watches, smart phones, smart glasses, activity tracking devices and other personal programmable devices which can be carried or worn by a user which may determine biometric data. Sensors can be embedded in areas users can frequent or objects frequently used, such as in walls, bed (e.g. sleep related sensors). Biometric sensors can capture data and values (measures) data inclusive of body movements and other physical motion data, gestures, facial expressions (smiles, grimaces, eye reactions and positioning and movements, etc.), auditory statements and outbursts and vocal tones and volumes, heartbeat, heartrate, respiration amounts or rates or constituent components, blood oxygen, motions, insulin levels, blood sugar levels, body temperatures, complexion coloring, etc., that may be indicative of an emotional state of the user (calm, upset, happy, sad, crying-emotional, etc.) from one or more camera (image), microphone (audio) and biometric sensors in wired or wireless circuit communication with the processor. Illustrative but not exhaustive examples of biometric sensors include cameras or other visual data scanners; microphones and other audio data sensors; wearable devices and sensors, such as smart watches, smart rings, fitness trackers and other wearable devices, and other devices located near enough to the user to acquire biometric data as a function of signal data received by their sensor components.

Aspects use smart glasses, smart watches, cameras and other wearable devices with outward-facing cameras to capture image data of the user activity, as well as biometric data relevant to a cognitive state of the user, and external cameras, such as used for video conferencing or generally monitoring image data within an environment occupied by the user, in order to thereby capture image data including user motion patterns and facial expressions. As previously alluded to, biometric sensors 232 may include microphones for capturing biometric audio data. Biometric audio data may include sound data from user utterances, speech, and other sound-generating activities. Examples of physiological biometric data acquired for a user by sensor components include heartbeat, heartrate, facial expression, intoxication, respiration amounts or rates or constituent components, blood oxygen, motions, insulin levels, blood sugar levels, etc. Other types of physiological biometric data that can be collected, include pulse, blood pressure, respiration, heart rate, heart rate variability, perspiration, temperature, and other physiological indicators of cognitive state. These and other biometric data can be determined passively, without contacting the user. Camera/vision 234 sensors may be useful in obtaining biometric image data generated by user activities. Biometric image or video data may be obtained from video and internal or external cameras in the environment of the user, embedded or otherwise communicatively coupled to devices described herein. Cameras can be internal to a smart phone, smart contact lens, eyeglass devices worn by a user or other person, internal or external to vehicles, smart appliances and/or smart devices, or cameras located externally to users at vantage points that capture user activities.

Biometric sensor 232 types include a variety of Internet of Things (IoT), Bluetooth®, or other wired or wireless devices that are personal to the user, and/or incorporated within environments (room, vehicle, home, office, vehicle, etc.) occupied by the user. Some environmental biometric signal sensors transmit a low-power wireless signal throughout an environment or space occupied by a user (for example, throughout a one- or two-bedroom apartment, inclusive of passing through walls), wherein the signal reflects off of the user's body, and the system 200 can analyze the reflected signals and determine and extract breathing, heart rate, sleep pattern or quality, gait and other physiological, biometric data of the user, as well as determine the cognitive state of the user as described herein.

In some embodiments, the cognitive state of the user can include that the user is in a non-agitated cognitive state (for example, only mildly upset, not angry, calm). For example, the user's heart rate may be calm, the user may be using happy, hopeful lexicography, their eye gaze does not wander, and contextually (e.g. via contextual and/or environmental sensors), the user is detected to be in a comfortable, and well-lit work environment, etc.) In some embodiments, the user may be detected to be in an agitated cognitive state (the user is angry, hot and sweaty, in an uncomfortable body position, with eyes darting, and a “scowling” facial expression, and speaking in angry tones). The cognitive state can include that the user is stressed, tired, alter, distracted, intoxicated, medicated, angry, and/or calm.

Sensors 220 can also be configured to monitor the control of the specific device the system 200 is part of, or monitor various aspects of the device and its performance. For example sensors 220 can be configured to detect one or more aspects controlled by control circuit 112 (with reference to FIG. 1).

During operation, cognitive state detection and response circuit 210 can receive information from various sensors 220, systems 258, and/or road traffic network 260 to determine whether a message has been received for which the sender should be identifies. Also, the driver, owner, and/or operator of the vehicle may manually trigger one or more processes described herein for detecting the sender of a message. Communication circuit 201 can be used to transmit and receive information between cognitive state detection and response circuit 210, sensors 152, cognitive state detection and response circuit 210 and/or systems 258. Also, sensors 152 and/or cognitive state detection and response circuit 210 may communicate with system elements 258 directly or indirectly (e.g., via communication circuit 201 or otherwise). Communication circuit 201 can be used to transmit and receive information between cognitive state detection and response circuit 210, one or more other system elements 258, but also other infrastructure or devices 260 (e.g. devices (e.g. mobile phones), systems, networks (such as a communications network and/or central server), and/or infrastructure. For example, via communication circuit 110, data relevant for determine the cognitive state of a user can be received, and one or more respective recommendations can be provided. In various embodiments, communication circuit 201 can be configured to receive data and other information from sensors 220 and/or systems 258 that is used in determining whether and how determine the sender of a message in a road traffic network. As one example, when a message is received from a an element of road traffic network 260, communication circuit 201 can be used to send an activation signal and/or activation information to one or more system elements 258 or sensors 120 for the vehicle to provide certain responsive information. For example, it may be useful for system elements 258 or sensors 120 to provide data useful in creating one or more hyper-graphs described herein. Alternatively, cognitive state detection and response circuit 210 can be continuously receiving information from system 258, sensors 120, other vehicles, devices and/or infrastructure (e.g. those that are elements of road traffic network 260). Further, upon determination of a cognitive state, communication circuit 201 can send a signal to other components of the system/device, infrastructure, or other devices based on the determination of the cognitive state. For example, the communication circuit 201 can send a signal to a system 258 element that indicates a control input for controlling the device based on the detected cognitive state of one or more users.

The examples of FIGS. 1 and 2 are provided for illustration purposes only as examples of systems and cognitive state detection and decision aid system 200 with which embodiments of the disclosed technology may be implemented. One of ordinary skill in the art reading this description will understand how the disclosed embodiments can be implemented with vehicle platforms.

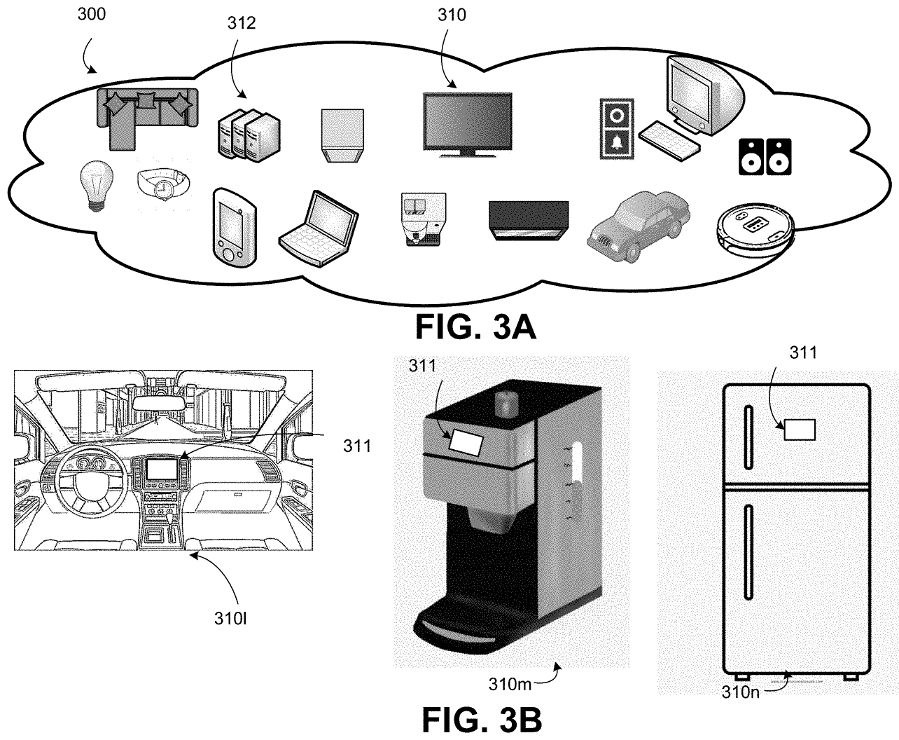

Referring now to FIG. 3A and FIG. 3B, FIG. 3A shows illustrative cloud or networked computing environment 300 including various devices with which aspects of the present disclosure can be implemented. As shown in FIG. 3A, cloud/network computing environment 300 includes one or more cloud computing nodes which can be implemented in at least part of connected devices 310. Connected device(s) 310 can be local computing devices used by cloud consumers, such as, for example, personal digital assistant (PDA) or cellular telephone, desktop computer, laptop computer, vehicles, wearable devices (such as clothing, watches, glasses), vending machines, coffee machines, speakers, coffee machine, vacuum, lighting systems (e.g. mood lighting), noise machines (e.g. for background noise), couches/beds/cushions (e.g. with adjustable softness), or refrigerator. Connected devices can include systems 100 and 200 shown in reference to FIG. 1 and FIG. 2. Example connected devices include vehicle 3101, coffee machine 310m, refrigerator 310n, and example graphical user interfaces 311 of systems 100/200 are shown in further detail in FIG. 3B. It can be understood that the various cognitive states as determined herein, as well as various respective recommendations, prompts, and/or messages can displayed at connected devices 310. It can also be understood that although various sensors described herein can be part of one device 310, the display (or other transmission to the user) of various recommendation(s), message(s), and/or prompts can be at another device 310.

In some embodiments, the graphical user interface 311 can be used to generate a prompt displaying the cognitive state of the user (e.g. predicted and/or actual) and/or an a visual indication thereof. Further, one or more recommendations can be displayed. It can also be understood that aspects of the connected device can be controlled by systems disclosed herein, based on the detected/predicted cognitive state of the user. As such, the display 311 can also for display of one or more states of the device (such as a control state indicating a present and/or future action of the device).

Continuing with reference to FIG. 3A, connected devices 310 can communicate with one another, including over a network. In an illustrative embodiment, the network is the Internet. The connected devices(s) 310 may be grouped (not shown) physically or virtually, in one or more networks, such as private, public or other networks/clouds, of any size or scope. Cloud/network computing environment 300 can offer infrastructure, platforms and/or software as services for which a cloud consumer do not need to maintain resources on a local computing device. It is understood that the types of devices 301 shown in FIG. 3A are intended to be illustrative only and that devices 310 and cloud computing environment 300 can communicate with any type of computerized device over any type of network and/or network addressable connection (e.g., using a web browser).

In addition to cloud computing embodiments, implementation of aspects of the present disclosure are not limited to a cloud computing environment. Rather, embodiments of the present invention are capable of being implemented in conjunction with any other type of computing environment. Computing environment 300 in the illustrative embodiment can include one or more server computers 312 and the network can interconnect the server computer(s) or data processing device(s) with one or more databases, and/or one or more client devices of connected devices 310. In other words, one or more devices 310 can be servers, and one or more other devices can be client devices in communication with the servers. The client device(s) may be continually or periodically connected to other client/server devices. The client device(s) may be able to access, provide, transmit, receive, and modify information over wired or wireless networks.

As previously alluded to with reference to FIG. 2, user devices can include aspects of cognitive state detection and decision aid system 200. For example, user devices can include respective sensors (such as GPS, camera, alcohol, vibration sensors), decision and control circuit (see circuit 203 with reference to FIG. 2), and user interfaces. Cognitive state detection and decision aid system 200, or aspects thereof, can be executed at a user device, for example by execution of one or more applications. In some embodiments, one or more sensors or other circuits of the user device (such as circuits shown in FIG. 1) can contribute to the determination of the cognitive state of a user. For example, as previously alluded to, various biometric sensors can be embedded in user devices (such as cameras, etc.). A microphone and/or processing component of the user device can be used to executive aspects of the conversation agent(s) described herein.

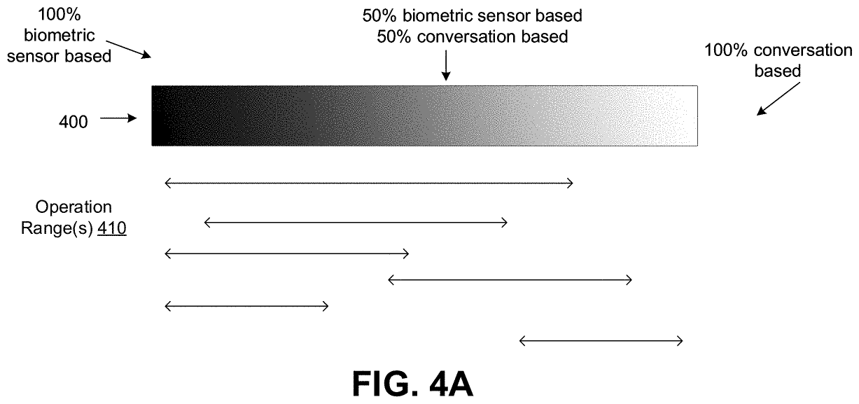

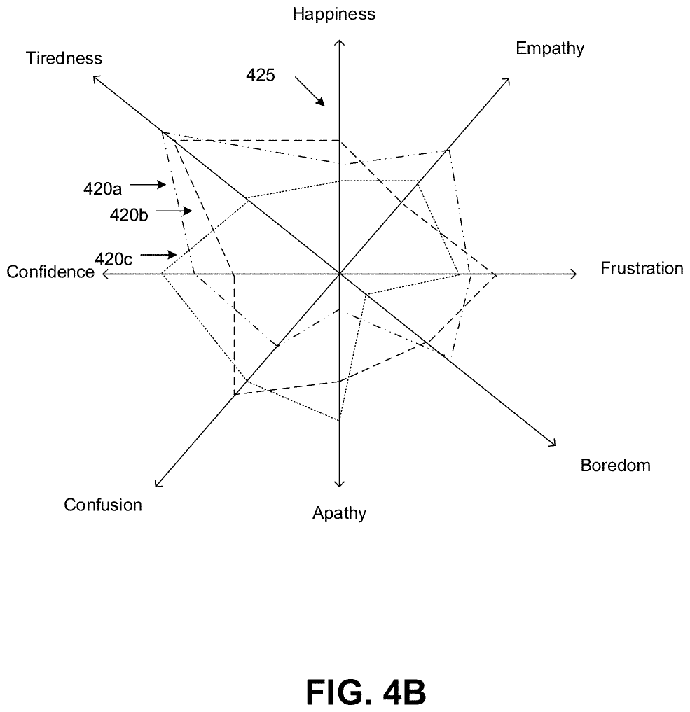

As previously alluded to, a cognitive state can be created from one or more biometric sensor based data, and/or by determination via a conversation agent. FIG. 4A illustrates a blending scale 400 for various sources for creation of a cognitive state, including via biometric sensors, and/or via a conversation agent. Also shown in FIG. 4A, are various operating ranges 410 for various implementations of example systems. Although the scale is shown as one dimensional, it can be understood that there can be one or more contributing factors to a determined cognitive state of a user, and as such the scale(s) can have one or more dimensions, and the operating ranges 410 can be multi-dimensional. In other words, a multi-dimensional scale can be created, wherein systems 100/200 can operate at one or more operational ranges of the scale. For example, systems 100/200 can base their recommendation based on user's use of one or more systems or devices (see various devices in FIGS. 3A-3B), other users' use of the system 100/200 (e.g. by collaborative filtering) or devices, based off of biometric sensing data, and/or by the conversation agent. The contributing factors can be weighted, and combined to form the cognitive state for the user for which recommendations and/or system 110/200 operation can be based.

One example scale shown in FIG. 4A, can be based off a premise that the cognitive state can be determined purely via one or more biometric sensors, or purely via determination by asking the user (e.g. by a conversation agent), and/or any combination of the two. As for asking the user, the conversation agent can be configured to indirectly and/or directly ask the user one or more questions, or otherwise converse with one or more users.

What a user says (e.g. in conversation) may not always correspond to how the user really feels. As such, a blending of biometric sensor sources as well as conversation agent, or other interaction sources, are useful for generation of the cognitive state. In some embodiments, data from the biometric sensors can be used to verify the data from the conversation agent, and/or reinforce the learning of the cognitive state. In some embodiments, the cognitive state of the user updates only when the cognitive state as determined from the biometric sensors matches (e.g. within 0.5%, 2%, within 5%, within 10%, with 15%, within 25%, within 30%, within 45%), the cognitive state as determined by the conversation agent or other interactions. In some embodiments, the cognitive state is determined by reinforcement learning, without needing to check the accuracy based on a subset of values.

As previously alluded to, cognitive states or cognitive states can change from time to time, and be different from user to user. FIG. 4B shows example cognitive states (420a, 420b, 420c), including across various dimensions, or with various contributing factors 425. The various dimensions or contributing factors are merely non-limiting examples. As described herein, various actions may be taken to minimize the cognitive load of a user. As described herein, minimizing the cognitive load of a user may mean that recommendations are tailored such that specific cognitive states are maintained and/or targeted. It can also be understood that various contributing factors to the cognitive state or cognitive state of a user can be weighted. For example, the values for various elements or dimensions of the cognitive state or cognitive state of a user may be different than how they are weighted. They may be weighted according to an importance (i.e. to a user, as reported or as determined), according to the user, according to frequency or degree/slope of changing (i.e. over time), according to values for other contributing factors, according to the number and/or type of interactions, according to values for the biometric data, etc. Cognitive states as disclosed herein may be dynamic and change over time, depending on the selection, and/or depending on one or more contextual factors (such as the environment). Some contributing factors may be so well linked or correlated, that they may be weighted the same or about the same, and/or weighted together. Others may be so inapposite, that they may be weighted poorly if they have specific values. In some embodiments, a first factor can be weighed as more important in contributing to the cognitive state than a second factor, unless the second factor has a specific level or value. For example, an individual's veracity or capacity for veracity may adjust they weighting for other contributing factors. In some embodiments, biometric sensor learned based factors can be weighted more than conversationally learned factors. In some embodiments, a value for one dimension of a user's cognitive state, can be weighed differently than another. In some embodiments, factors learned from biometric sensors can be weighted more than factors learned by correspondence with the user, unless one or more values thereof are below, at, or above a specific value.

In some embodiments, the user may have interactions with the system 100/200 (e.g. via the conversation agent). In some embodiments, it may be important for the system 100/200 to retrieve data in the context of repeated interactions with the system 100/200. In some embodiments, the repeated interactions, in various contexts (e.g. contextual environments, times of the day), can allow for increased precision and/or accuracy in generating the cognitive states. The number of, outcomes for, and types of interactions with the system may determine the blending of sources of information (see blending of biometric sensor based data with interactions based data with respect to FIG. 4A, and the various operating ranges 410). The dimension of a cognitive state than needs to be learned (e.g. agitation, tiredness, happiness, openness, relaxation), may change the operating ranges 410 and/or the weights for the sources of information. In the context of repeated interactions, the number of repetitions may adjust the operating range 410 and/or the various weights for the sources of information. In the context of repeated and/or varied interactions, a baseline cognitive state can be created. Further, one or more out of bounds state(s) can be created. In some embodiments, the profile corresponding to one or more cognitive states can be created (e.g. if the user is tired, agitated, relaxed, happy, confident, etc.).

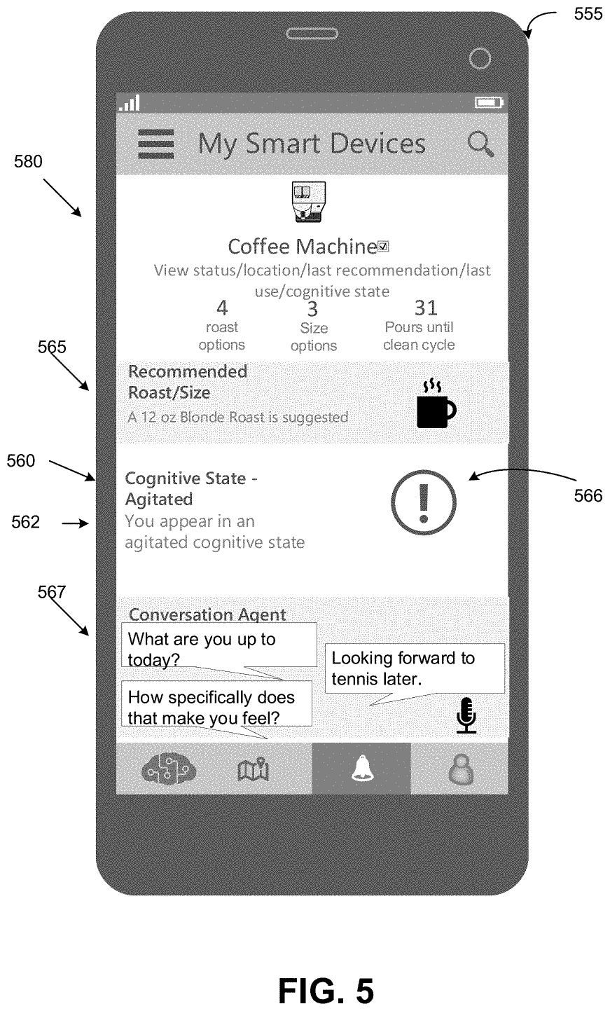

It can also be understood that the various cognitive states as determined herein, as well as various recommendations, prompts, or messages, can displayed at a user device. As previously alluded to, one example connected device in which aspects of the present disclosure can be implemented, is a user device. Referring now to FIG. 5, FIG. 5 illustrates a graphical user interface at a user device 555. User device 555 can allow for display (and/or by other visual, audible, and/or haptic or other feedback) of one or more cognitive states, recommendations, indications, alerts, or prompts disclosed herein. For example, a user device 555 can be a smart phone, laptop, augmented reality glasses, and/or one or more other connect devices described herein (see, for example with reference to FIGS. 3A/3B).

User device 555 (or application thereof), can allow for display and selection of one or more devices, adding new devices, display of one or more available services, display of notifications (including display of one or more cognitive states 560 and/or recommendations 565), display/editing of user profiles and/or biometric profiles, and allow for user interaction with a conversation agent.

By an application executed at a user device 555, the cognitive state, the status of and/or respective recommendations for one or more devices can be displayed at an interface of the user device 555. For example, one or more determined and/or predicted cognitive state(s) of the user 560 can be displayed for the user, including across one or more specific dimensions for the cognitive state. The displayed cognitive state 560 can be context specific (i.e. specific to the context of the selected device). The cognitive state can be displayed before, during, and/or after, a user expects to receive a recommendation 565 based on the cognitive state. The recommendation can include any type of visual and/or audio indication of a recommendation. By narrowing the scope of (e.g. number of) options related to user decisions, the recommendation can allow for reducing the stress and/or cognitive load associated with decision making.

The displayed cognitive state can include one or more visual indications, with the indications corresponding to one or more levels or values for the cognitive state. In some embodiments, the cognitive state can be displayed as shown with reference to FIG. 4B. The cognitive state can include one or more associated prompts 562. Prompts 562 can include affirmative and/or contextual statements regarding the specific cognitive state (e.g. “you appear tired,” “you appear agitated.”). Prompts 562 can also include one or more questions for which responses from the user are expected. For example, they can include prompts from the aforementioned conversation agent (e.g. “how do you feel today?” “what is 2 times 3?” “do you remember what is on your agenda for today?”). As such, one or more prompts 567 from a conversation agent can be displayed and/or vocalized. Prompts 567 can allow for entry of one or more responses to the prompts (e.g. by microphone and/or by text based entry). The prompts 567 can allow for determination of the cognitive state of the user. The prompts can be conversational prompts, such as open-ended prompts, greetings, target questions, probing questions, misdirection questions, open-ended questions.

One or more indications, alerts, or prompts can be displayed before a user is predicted to use a device. The status 580 (e.g. connection status, one/of status, location, battery level, options from which recommendations can be generated) can be displayed.

At the interface, one or more devices with cognitive based recommendations can be selected from (or automatically displayed). The device respective status, contextual cognitive state (i.e. in the context of use of the specific device), and/or recommendations can be displayed. One or more recommendations 565 can be displayed. The recommendations can be based on one or more options available for selection, can be related to the specific device, and can be based on a detected and/or predicted cognitive state of the user. The recommendation can be based on location. For example, a location of the user can be used to establish a need fora device for which a cognitive state based recommendation would be useful. A mapping function can include the location of one or more connected devices, including visual display of the one or more devices, including with respect to the user.

In some embodiments, a contextual cognitive state 560 may change depending on a context. For example, a user may be predicted to make a specific decision (automatically or by selection of a user). A cognitive state may be determined and a recommendation based on the cognitive state can be determined. Despite this, the user may make a selection outside of the recommendation. The system 100/200 may then analyze the user's cognitive state (for the first time, or again), and determine that the user is in a different cognitive state. A recommendation 650 may then adjust based on the updated cognitive state. In some embodiments, the recommendation can be configured to minimize the cognitive load of the user. As such, the recommendation can be configured to minimize the number of selections available to the user, and/or by the user making a selection, adjust the cognitive state or profile of the user. In some embodiments, an outcome of a selection of a prior recommendation can allow for providing feedback to the system 100/200. As such, the recommendation may be configured to allow the user to try a new selection or possible option. In some embodiments, the recommendation may be selected so as to move an individual towards not trying new things. In some embodiments, the recommendation can be configured to change or maintain a habit, and/or allow the system to learn if the user may like one or more other things. In some embodiments, it can be a goal of the system to not force the user to try something new, but merely lessen the load of making decisions.

Although a user interface is displayed, an administrator interface can also be included. Administrator interfaces can be available to users. For example, limits can be set on the extent to which the device automatically modifies the choice set. An administrator interface may allow for adjusting of the sources of data useful in determining the cognitive state of the user. One or more operating states (with reference to discussion of FIG. 4A) and/or cognitive states (e.g. in time series) can be viewed and/or adjusted. For example, the sources of data can be any blend of biometric sensor based (including specificity of the type of biometric sensor) and/or conversation based. Further, one or more training data, weights, and/or biases for the sources and/or values for the data maybe adjusted. Current and/or prior interaction and/or biometric sensor data can be viewed.

FIG. 6A and FIG. 6B shows method 600 and method 620 respectively, which can be performed for determining a cognitive load of a user and/or aiding in a user's decision making process. The methods 600, 620 can be performed at cognitive state detection and decision aid system 200 (e.g. a decision and control circuit 203) as shown in FIG. 2, system 100 with reference to FIG. 1. It can be understood that methods 600, 620 can be performed at one or more of the devices shows with reference to FIG. 3A, FIG. 3B, and FIG. 5.

The steps shown are merely non-limiting examples of steps that can be included for determining and recommending based on a cognitive state of a user. The steps shown in method 600 and method 620 can include and/or be included in (e.g. executed as part of) one or more circuits or logic described herein. It can be understood that the steps shown can be performed out of order (i.e. a different order than that shown in FIGS. 6A and 6B), and with or without one or more of the steps shown. These steps can also repeat, for example for performing of steps according to updated information. The steps can also be performed according to data of various time points in a time series.

Referring again to FIG. 6A, FIG. 6A shows method 600 for determining a cognitive state of a user and/or aiding in a user's decision making process. Method 600 can include step 602 for receiving and/or updating data. The data can be present values (e.g. from sensors 220, system 258, and/or from connected elements 260 (e.g. from databases, user devices or other users) shown with reference to FIG. 1 and FIG. 2). Received data can be from one or more devices (see FIGS. 3A-3B and FIG. 5). Received data can be based on at least one of biometric sensed data and/or based on conversations with a conversational agent (see with reference to FIG. 4A, NLP circuit 117 and vocalization circuit 116 with reference to FIG. 1). In some embodiments, data can be received by a cognitive state detection circuit 114 that has two primary modes of functioning. In a first mode when a camera is available, the system can leverage computer vision and artificial intelligence technology to identify the cognitive state based on at least face, eye, and body features extracted from the image and/or video, and wherein the video data includes information from a video interview wherein the system asks the user multiple questions and then determines the next action for the user. In a second mode, when a camera is not available, a built-in conversational agent that utilizes a microphone and speaker and can engage in a brief dialogue with the user by asking multiple questions. Receiving data at step 602 can include extracting one or more data from received responses to the multiple questions (or generally responses to one or more prompts). For example, the data can be extracted by NLP circuit 117. Receiving data at step 602 can include receiving a user identifier (see with reference to FIG. 1). The data received can be useful in determining the cognitive state of one or more users.

Method 600 can include step 604 for learning the cognitive state of one or more users. Learning the cognitive state of one or more users can include learning cognitive states including values for various contributing factors (see generally FIG. 4B). Learning the cognitive state can include learning one or more weights for the various contributing factors, and/or biasing towards data received (see step 602) from specific sources (see with reference to FIG. 4A).

Method 600 can include step 606 for recommending a next action for the user to take (see with reference to recommendation circuit 115 in FIG. 1). The action can be with reference to one or more devices in a network of connected devices. As previously mentioned one or more recommendations, suggestions, messages, and/or prompts can be provided by systems described herein. The recommended action can be provided as an indication of subset of available sets of functions, options, selections related to one or more devices, software components, or services described herein.

Further, systems described herein can control one or more aspects of the system based on recommendation. Said differently, one or more aspects of the system can be controlled so as to act upon recommendations generated based on the detected cognitive states described herein. As such, step 606 can include generating and/or adjusting a control signal. The control signal (i.e. the adjusting/generation thereof) can be based on the learned cognitive state (i.e. at step 604). The control signal can be an input signal for one or more components of systems described herein (see control systems 223). For example, actuation signals can be provided with respect to actuators of devices described herein (such as vehicles, machinery, etc.). The control signal can be adjusted based on one or more operational parameters for the device (see operational parameter database 133). Devices described herein can be configured with two or more operational configurations (e.g. devices can be configured to provide selections between one or more operational settings). The control signal can allow for selection from a subset of the two or more operational configurations of the devices, based on generated recommendations described herein. Again, the recommendation and/or control signal can be based on the detective cognitive states.

FIG. 6B shows another method 620 for determining a cognitive state of a user and/or aiding in a user's decision making process. Method 620 can include step 622 for receiving and/or updating data. The data can be present values (e.g. from sensors 220, system 258, and/or from connected elements 260 (e.g. from databases, user devices or other users) shown with reference to FIG. 1 and FIG. 2). Received data can be from one or more devices (see FIGS. 3A-3B and FIG. 5). Received data can be based on at least one of biometric sensed data and/or based on conversations with a conversational agent (see with reference to FIG. 4A, NLP circuit 117, and vocalization circuit 116 with reference to FIG. 1). In some embodiments, data can be received by a cognitive state detection circuit 114 that has two primary modes of functioning. In a first mode, when a camera is available, the system can leverage computer vision and artificial intelligence technology to identify the cognitive state based on at least face, eye, and body features extracted from the image and/or video. The video data can include information from a video interview wherein the system asks the user multiple questions and then determines the next action for the user. In a second mode, when a camera is not available, a built-in conversational agent that utilizes a microphone and speaker and can engage in a brief dialogue with the user by asking multiple questions. Receiving data at step 622 can include extracting one or more data from received responses to the multiple questions (or generally responses to one or more prompts). For example, the data can be extracted by NLP circuit 117). Receiving data at step 622 can include receiving a user identifier (see with reference to FIG. 1). The data received can be useful in determining the cognitive state of one or more users.

Method 620 can include step 624 for learning the cognitive state of one or more users based on one or more cognitive state models (see cognitive state models 136 with reference to FIG. 1). Learning the cognitive state of one or more users can include learning cognitive states including values for various contributing factors to the cognitive state (see generally FIG. 4B). Learning the cognitive state can include learning one or more weights for the various contributing factors, and/or biasing towards data received (see step 622) from specific sources (see with reference to FIG. 4A). A cognitive state model can include a mapping between values for one or more contributing factors to the cognitive state, the sources for the data, and one or more recommendations. For example, for the same values of contributing factors, the mapping can be different for recommending first type of user action, than recommending second type of user action.

Method 620 can include step 626 for recommending a next action for the user to take (see with reference to recommendation circuit 115 in FIG. 1). The action can be with reference to one or more devices in a network of connected devices. As previously mentioned one or more recommendations, suggestions, messages, and/or prompts can be provided by systems described herein. The recommended action can be provided as an indication of subset of available sets of functions, options, configurations, and/or selections related to one or more devices, software components, or services described herein.

Further, systems described herein can control one or more aspects of the system based on recommendation. Said differently, one or more aspects of the system can be controlled so as to act upon recommendations generated based on the detected cognitive states described herein. As such, step 626 can include (in addition or alternatively) generating and/or adjusting a control signal. The control signal (i.e. the adjusting/generation thereof) can be based on the learned cognitive state (i.e. at step 624). The control signal can be an input signal for one or more components of systems described herein (see control systems 223). For example, actuation signals can be provided with respect to actuators of devices described herein (such as vehicles, machinery, etc.). The control signal can be adjusted based on one or more operational parameters for the device (see operational parameter database 133). Again, the recommendation and/or control signal can be based on the detective cognitive state(s) of user(s).

Method 620 can include step 628 for receiving second data. The second data can be of the same or different form or source as received at step 622. Method 620 can include step 630 for updating one or more training sets, baselines, circuits, models, machine learning models described herein based on the received second data (i.e. at step 628). For example, the weights for various factors can be adjusted.

With reference to methods 600, 620, it can be understood that one or more data (e.g. the data from steps 602, 622, 628) can be updated based on the determination of one or more aspects of cognitive state or state, an outcome of adjusting the control input based on the cognitive state (see step 606, 626), and/or determining the cognitive state at steps 604, 624. It can also be understood that one or more training sets, baselines, circuits, models, machine learning models described herein can be adjusted and/or updated. For example, the weights for various factors can be adjusted.

As used herein, the terms circuit, system, and component might describe a given unit of functionality that can be performed in accordance with one or more embodiments of the present application. As used herein, a component might be implemented utilizing any form of hardware, software, or a combination thereof. For example, one or more processors, controllers, ASICs, PLAs, PALs, CPLDs, FPGAs, logical components, software routines or other mechanisms might be implemented to make up a component. Various components described herein may be implemented as discrete components or described functions and features can be shared in part or in total among one or more components. In other words, as would be apparent to one of ordinary skill in the art after reading this description, the various features and functionality described herein may be implemented in any given application. They can be implemented in one or more separate or shared components in various combinations and permutations. Although various features or functional elements may be individually described or claimed as separate components, it should be understood that these features/functionality can be shared among one or more common software and hardware elements. Such a description shall not require or imply that separate hardware or software components are used to implement such features or functionality.

Where components are implemented in whole or in part using software, these software elements can be implemented to operate with a computing or processing component capable of carrying out the functionality described with respect thereto. One such example computing component is shown in FIG. 1. After reading this description, it will become apparent to a person skilled in the relevant art how to implement the application using other computing components or architectures.

In this document, the terms “computer program medium” and “computer usable medium” are used to generally refer to transitory or non-transitory media. Such media may be, e.g., storage medium 110, storage devices 120 and channel 154. These and other various forms of computer program media or computer usable media may be involved in carrying one or more sequences of one or more instructions to a processing device for execution. Such instructions embodied on the medium, are generally referred to as “computer program code” or a “computer program product” (which may be grouped in the form of computer programs or other groupings). When executed, such instructions might enable the computing component (e.g. processor 104) to perform features or functions of the present application as discussed herein.

As described herein, vehicles can be flying, partially submersible, submersible, boats, roadway, off-road, passenger, truck, trolley, train, drones, motorcycle, bicycle, or other vehicles. As used herein, vehicles can be any form of powered or unpowered transport. Obstacles can include one or more pedestrian, vehicle, animal, and/or other stationary or moving objects. Although roads are references herein, it is understood that the present disclosure is not limited to roads or to 1d or 2d traffic patterns.

The term “operably connected,” “coupled”, or “coupled to”, as used throughout this description, can include direct or indirect connections, including connections without direct physical contact, electrical connections, optical connections, and so on.

The terms “a” and “an,” as used herein, are defined as one or more than one. The term “plurality,” as used herein, is defined as two or more than two. The term “another,” as used herein, is defined as at least a second or more. The terms “including” and/or “having,” as used herein, are defined as comprising (i.e. open language). The phrase “at least one of . . . and . . . .” as used herein refers to and encompasses any and all possible combinations of one or more of the associated listed items. As an example, the phrase “at least one of A, B, or C” includes A only, B only, C only, or any combination thereof (e.g. AB, AC, BC or ABC).

Aspects herein can be embodied in other forms without departing from the spirit or essential attributes thereof. Accordingly, reference should be made to the following claims, rather than to the foregoing specification, as indicating the scope hereof. While various embodiments of the disclosed technology have been described above, it should be understood that they have been presented by way of example only, and not of limitation. Likewise, the various diagrams may depict an example architectural or other configuration for the disclosed technology, which is done to aid in understanding the features and functionality that can be included in the disclosed technology. The disclosed technology is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the technology disclosed herein. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order, and/or with each of the steps shown, unless the context dictates otherwise.

Although the disclosed technology is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the disclosed technology, whether or not such embodiments are described and whether or not such features are presented as being a part of a described embodiment. Thus, the breadth and scope of the technology disclosed herein should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed in multiple groupings or packages or across multiple locations.

Additionally, the various embodiments set forth herein are described in terms of exemplary block diagrams, flow charts and other illustrations. As will become apparent to one of ordinary skill in the art after reading this document, the illustrated embodiments and their various alternatives can be implemented without confinement to the illustrated examples. For example, block diagrams and their accompanying description should not be construed as mandating a particular architecture or configuration.

What is claimed is:

1. A computer implemented method for detection of a cognitive state of a user, the method comprising:
receiving, by a perception circuit comprising at least one sensor, at least one biometric sensor data;
generating a first signal for a user interface, the signal based on one or more conversational prompts;
generating, by a processing component, a prediction of a cognitive state for a user based on the at least one biometric sensor data;
generating a recommendation based on the predicted cognitive state for the user, and;
providing, a second signal for the user interface, the second signal comprising an indication of the generated recommendation.

2. The method of claim 1, wherein the recommendation comprises a subset of a set of options, wherein the set of options comprise possible operational configurations of a device.

3. The method of claim 1, further comprising: generating a control signal, the control signal configured to control an operation of a device based on the generated recommendation.

4. The method of claim 1, wherein the recommendation is a common recommendation for multiple users, based on predictions of respective cognitive states for multiple users.

5. The method of claim 1, wherein the user interface comprises a vocalization circuit.

6. The method of claim 1, further comprising receiving, by the user interface, a response to the one or more conversational prompts, wherein the cognitive state for the user is further generated based on the content of the received response.

7. The method of claim 6, further comprising receiving, by the perception circuit comprising at least one sensor, a second biometric sensor data, and;
updating a cognitive state machine learning model based on the second biometric sensor data and the generated recommendation.

8. The method of claim 1, further comprising receiving a user identifier, and only generating the prediction if the user identifier matches the biometric sensor data.

9. A system, comprising:
at least one memory storing machine-executable instructions; and
at least one processor configured to access the at least one memory and execute the machine-executable instructions to:
detect an availability of a camera based sensor;
generating a first a signal for a user interface, the signal based on one or more conversational prompts;
if the availability indicates a camera based sensor is available, determine the cognitive state of a user based on features extracted from signals from the camera based sensor;
if the availability indicates a camera based sensor is not available, receiving, by at least one other sensor, at least one biometric sensor data;
generate, by a processing component, a prediction of a cognitive state for a user based on the received at least one biometric sensor data;
generate a recommendation based on the predicted cognitive state for the user, and;
provide a second signal for the user interface, the second signal comprising an indication of the generated recommendation.

10. The system of claim 9, wherein if the availability indicates the camera based sensor is available, the first signal for the user interface comprises a video conversational prompt, and if the availability indicates the camera based sensor is not available, the first signal for the user interface comprises an audio based vocalized question.

11. The system of claim 10, wherein the recommendation comprises a subset of a set of options, wherein the set of options comprise possible operational configurations of an operational component of the system, and wherein the operational component of the system comprises at least a scheduling component, a home appliance operational controller, or a navigation system.

12. The system of claim 10, further comprising an operational component configured with two or more operational configurations, wherein the at least one processor is configured to access the at least one memory and execute the machine-executable instructions to generate a control signal, the control signal configured to control an operation of the operational component according to a subset of the two or more operational configurations based on the generated recommendation.

13. The system of claim 10, wherein the recommendation is a common recommendation for multiple users, based on predictions of respective cognitive states for multiple users.

14. The system of claim 10, wherein the at least one processor is configured to access the at least one memory and execute the machine-executable instructions to receive a response input signal based on a user response to the one or more conversational prompts, and wherein the cognitive state for the user is further generated based on the response input signal.

15. The system of claim 10, wherein the predicted cognitive state for the user was generated based on a cognitive state machine learning model, and wherein the processor is configured to access the at least one memory and execute the machine-executable instructions to receive subsequent biometric sensor data from the at least one other sensor, and update a cognitive state machine learning model based on the subsequent biometric sensor data and the generated recommendation.

16. The system of claim 10, wherein the processor is configured to access the at least one memory and execute the machine-executable instructions to receive a user identifier, wherein the predicted cognitive state is only generated if the user identifier matches the biometric sensor data.

17. A system comprising:
at least one memory storing machine-executable instructions; and
at least one processor configured to access the at least one memory and execute the machine-executable instructions to:
receive, by a perception circuit comprising at least one sensor, at least one biometric sensor data;
generate a first a signal for a user interface, the signal based on one or more conversational prompts;
generate, by a processing component, a prediction of a cognitive state for a user based on the received at least one biometric sensor data;
generate a recommendation based on the predicted cognitive state for the user, and;
provide, a second signal for the user interface, the second signal comprising an indication of the generated recommendation.

18. The system of claim 17, wherein the recommendation comprises a subset of a set of options, wherein the set of options comprise possible operational configurations of a device.

19. The system of claim 17, wherein the at least one processor is configured to access the at least one memory and execute the machine-executable instructions to generate a control signal, the control signal configured to control an operation of a device based on the generated recommendation.

20. The method of claim 17, wherein the recommendation is a common recommendation for multiple users, based on predictions of respective cognitive states for multiple users.
